(19) United States
(12) Patent Application Publication
(10) Pub. No.: US 2023/0406804 A1
(43) Pub. Date: Dec. 21, 2023

(54) PROTEIN KINASE INHIBITORS

(71) Applicants: Xavier University of Louisiana, New Orleans, LA (US); The Administrators of the Tulane Educational Fund, New Orleans, LA (US)

(72) Inventors: Jayalakshmi SRIDHAR, New Orleans, LA (US); Frank JONES, New Orleans, LA (US); Cheryl STEVENS, Bowling Green, KY (US)

(21) Appl. No.: 18/451,630

(22) Filed: Aug. 17, 2023

Related U.S. Application Data
(63) Continuation of application No. 17/284,432, filed on Apr. 9, 2021.
(60) Provisional application No. 62/744,795, filed on Oct. 12, 2018.

(51) Int. Cl. C07C 50/12

(52) U.S. Cl. CPC C07C 50/12

(57) ABSTRACT

The present disclosure relates to compounds that act as protein kinase inhibitors, and the synthesis of the same. Further, the present disclosure teaches the utilization of such compounds in a treatment for proliferative diseases, including cancer, particularly breast cancer, and especially ER+ and/or HER2+ breast cancer.

### CROSS REFERENCE TO RELATED APPLICATIONS

This application is a continuation application of U.S. patent application Ser. No. 17/284,432, filed Apr. 9, 2021, which is a National Stage entry of International Application No. PCT/US2019/055330, filed Oct. 9, 2019, which claims priority to U.S. Provisional Application No. 62/744,795, filed on Oct. 12, 2018, the contents of which are incorporated herein by reference in its entirety.

### STATEMENT REGARDING FEDERALLY SPONSORED RESEARCH OR DEVELOPMENT

This invention was made with government support under W81XWH-11-1-0105 awarded by Department of Defense (DOD). The government has certain rights in the invention.

### FIELD OF INVENTION

The present disclosure relates to compounds that inhibit protein kinase and reverse therapeutic resistance in cancer cells. The disclosure also relates to pharmaceutical compositions comprising these small molecule protein kinase inhibitors, and methods for using the same for treatment of tyrosine kinase-dependent diseases and conditions, including cancers.

### BACKGROUND

### 1. Field

The present disclosure relates to compounds having, for example, activities as protein kinase inhibitors, and methods for making the same. The disclosure also relates to pharmaceutical compositions comprising these protein kinase inhibitors, and methods for using the same for treatment of protein kinase-dependent diseases/conditions, including cancer, neoangiogenesis, atherosclerosis, diabetic retinopathy or inflammatory diseases, in mammals. Cancers include breast cancer, non-small cell lung cancer, renal carcinoma, ovarian cancer, etc.

The compounds described here can provide effective therapy for breast cancers, especially estrogen receptor positive or “ER+” breast cancers or HER2-positive breast cancers, as, for example, the first line adjuvant treatment regimen, or in the second-line setting as treatment for patients with disease progression after not being responsive to or has become resistant to other treatments, such as trastuzumab, lapatinib, tamoxifen therapy, or combinations of such therapies with other anticancer agents.

### 2. Description of Related Art

There are over three million women living in the U.S. with breast cancer and the American Cancer Society estimates that 332,630 Americans will be diagnosed with breast cancer in 2018 and 41,400 will die from breast cancer in 2018 [1]. A 2015 National Cancer Institute study projected U.S. cancer diagnoses to increase from 283,000 in 2011 to 441,000 in 2030—a more than 50% increase [2]. Worldwide, every year over 1.5 million people are diagnosed with breast cancer and over 500,000 people die from breast cancer [3].

Tyrosine kinases are a class of enzymes that catalyze the transfer of the terminal phosphate of adenosine triphospate to tyrosine residues in protein substrates. Tyrosine kinases are believed, by way of substrate phosphorylation, to play critical roles in signal transduction for a number of cell functions. Though the exact mechanisms of signal transduction is still unclear, tyrosine kinases have been shown to be important contributing factors in cell proliferation, carcinogenesis and cell differentiation. Aberrant expression of protein kinases and their dysfunction are associated with several subtypes of breast cancer, and protein kinases are used as biomarkers for aggressive breast cancer.

Accordingly, inhibitors of these tyrosine kinases are useful for the prevention and treatment chemotherapy of proliferative diseases dependent on these enzymes. Receptor tyrosine kinase (RTK) inhibitors have become an area of research interest because they are targeted to cancer cells (they don't damage healthy cells in addition to cancer cells, so they result in fewer side effects), research shows that RTKs (including HER2 and EGFR) have roles in aggressive cancers and in drug resistance, indicating potential for RTK inhibitors to overcome current treatment limitations [4].

There are three major molecular subtypes of breast cancer based upon the presence or absence of two receptors. Accordingly, estrogen receptor (ER) expressing tumors are referred to as ER+, and the Human epidermal growth factor 2 expressing HER2+ breast cancers. These types of cancers are treated with targeted therapies that block/inhibit the growth of cancer by interfering with the activity of specific receptor that is responsible for the disease condition.

A significant amount of breast cancers are ER-positive [5]. In estrogen receptor positive breast cancer, tamoxifen is the most common treatment, but 20-30% of patients are resistant to tamoxifen [6]. 20-30% of all breast cancers are HER2-positive [7]. Research shows that an oncogenic isoform of HER2, HER2Δ16, which is expressed in over 30% of ERα-positive cancers, is involved in estrogen-independent growth and de-novo resistance (resistance from the start, not over time) to Tamoxifen. 70% of patients with HER2-positive tumors demonstrate intrinsic or secondary resistance to trastuzumab.

As many as 20% of breast cancers are triple negative [8]. In triple-negative breast cancer (HER2-negative, ER-negative, and progesterone receptor-negative), which is the most aggressive subtype, the tyrosine kinase epidermal growth factor receptor (EGFR) is frequently overexpressed [9]. EGFR inhibitors have been approved for other types of cancer and lapatinib (a dual EGFR/HER2 inhibitor) is approved for HER2+ breast cancer, but there are currently no FDA-approved EGFR-targeting medications for triple negative breast cancer [10].

The last decade has seen significant advances in breast cancer therapies for HER2 positive tumors with the clinical use of trastuzumab and trastuzumab in combination with other chemotherapeutics leading to increased response rates [11]. Despite this recent advance, ˜70% of patients with HER2 positive tumors demonstrate intrinsic or secondary resistance to trastuzumab, current breast cancer therapy for HER2 positive tumors [12]. Among the several mechanisms of resistance proposed for HER2-targeted therapy with trastuzumab, the specific implicating factors include the presence of the oncogenic isoform HER2Δ16, presence of truncated HER2 (p95HER2), and HER2 dimerization status. Significantly, the trastuzumab-resistant oncogenic HER2Δ16 isoform has been found to be preferentially expressed in aggressive metastatic breast cancer. It was recently demonstrated that expression of HER2Δ16, but not wild-type HER2, in ERα-positive breast tumor cells promotes estrogen-independent growth and de novo resistance to tamoxifen therapy. Many other protein kinases (HER2, HER3, IGFIR, AKT, SRC, and PI3K-mTOR) are also involved in the development of resistance to standard treatment through downstream/upstream/co-activation, signal modulation and incomplete inhibition. Thus, a significant number of HER2-positive breast cancers are eventually relapsing which suggests that the HER2-targeting therapies can overtime be resisted. The dual EGFR/HER2 kinase inhibitor lapatinib has shown promising clinical results, but its limitations have also lead to resistance and activation of tumor survival pathways.

Therefore, there is a market need for more potent breast cancer drugs that do not encounter resistance or drugs that can reverse resistance to currently used treatments. Targeting protein kinases using small molecule protein kinase inhibitors would be a very effective strategy for reversing therapeutic resistance in breast cancer as tyrosine kinase inhibitors may be more effective in treating HER2-positive breast cancer, due to their ability to block downstream signaling pathways in p95HER2, HER2Δ16, and full length HER2.

### BRIEF SUMMARY

The present disclosure relates generally to novel compounds and compositions useful for the inhibition of tyrosine kinase; compounds, intermediates, and methods of making such compounds and compositions; methods of using such compounds and compositions; pharmaceutical compositions comprising such compounds and compositions; and methods of using such pharmaceutical compositions, among other things.

In an embodiment, the present invention provides derivatives of 2-methylnaphthalene-1,4-dione (formula (I)), or a stereoisomer or pharmaceutically acceptable salt thereof.

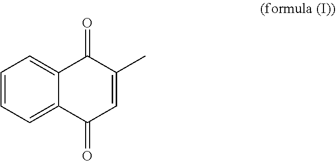

1,4-dione-naphthelene is a basic unit of Vitamin K, but since, for example, the naphthoquinone ring is not present in Vitamin K as a basic submit, these vitamins do not suggest the claimed compounds.

In another embodiment, the present invention provides a compound of formula (II) or a stereoisomer or pharmaceutically acceptable salt thereof:

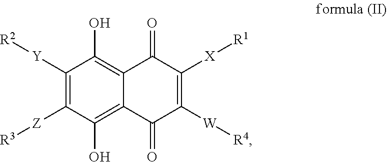

wherein:
W, X, Y and Z independently represent hydrogen, —C(R⁵)—, —O—, —S—, —CH₂O—, CH₂S—, —(CH₂)₂O—, —NR⁶—, —NR⁶CH₂—, —CH₂NR⁶—, —NR⁶CO—, —CONR⁶—, —N═N—, —NH—CO—NH—, —NH—CS—NH—, —CO—O—, CO—O—CH₂—, —SO₂NH—, —NH—SO₂—, —CR⁴═CR⁴—, —C≡C—, —O—CH₂—CO—, —OCH₂CH₂O—, —CH(OH)—, or —NO₂ bridging groups;
R⁵ independently represent hydrogen, halogen, C₁₋₆ alkyl, C₁₋₆ alkenyl, C₁₋₆ alkoxy, C₁₋₆ haloalkyl, haloC₁₋₆ alkoxy, —COOH, —CONH₂, —COC₁₋₆ alkyl, O—C₁₋₆ alkyl, NH₂, NH—C₁₋₆ alkyl, or —S C₁₋₆ alkyl groups; and
R¹, R², R³, R⁴ and R⁶ independently represent hydrogen, halogen, aryl, C₃₋₈ cycloalkyl, monocyclic or bicyclic heterocyclyl, monocyclic or bicyclic heteroaryl, wherein the aryl, heteroaryl or heterocyclyl groups may be optionally substituted by one or more R⁴ groups.

In a further embodiment, at least one of W, X, Y, or Z is methyl, and R¹, R², R³, and R⁴ are hydrogen.

In a further embodiment,
Y, X, and W are independently selected from H or —C(R⁵)—;
Z is hydrogen;
R¹, R², R³, and R⁴ are independently selected from hydrogen or halogen, and
R⁵ is hydrogen.

In a further embodiment,
Y X, and W are independently selected from hydrogen or methyl;
Z is hydrogen; and
R¹, R², R³, and R⁴ are independently selected from hydrogen or halogen.

In a further embodiment, the compound of formula (II) is 5,8-dihydroxy-2-methylnaphthalene-1,4-dione:
wherein W, Y, and Z are hydrogen, X is methyl, and R¹, R², R³, and R⁴ are hydrogen.
   An exemplary scheme for synthesizing the compound of Formula (II) is shown in FIG. 1.

In a further embodiment, the compound the formula (II) is 5, 8-dihydroxy-6-methylnaphthalene-1,4-dione:
wherein Y is methyl, W, X, and Z are hydrogen, and R¹, R², R³, and R⁴ are hydrogen.
   An exemplary scheme for synthesizing the compound of Formula (II) is shown in FIG. 1.

In a further embodiment, the compound of formula (II) is 5,8-dihydroxy-2,7-dimethylnaphthalene-1,4-dione, wherein Y is methyl, X is methyl, W and Z are hydrogen, and R¹, R², R³, and R⁴ are hydrogen. An exemplary scheme for synthesizing the compound of Formula (II) is shown in FIG. 1.

In a further embodiment, the compound of formula II is 5,8-dihydroxy-2,6-dimethylnaphthalene-1,4-dione, wherein: Y is methyl, X and Z are hydrogen, W is methyl, and R¹, R², R³, and R⁴ are hydrogen. An exemplary scheme for synthesizing the compound of Formula (II) is shown in FIG. 1.

In a further embodiment, the compound of formula II is 2-(bromomethyl)-5,8-dihydroxynaphthalene-1,4-dione, wherein: W, Y, and Z are hydrogen, X is CH₂—, R¹ is Br, and R², R³, and R⁴ are hydrogen. An exemplary scheme for synthesizing the compound of Formula (II) is shown in FIG. 1.

In a further embodiment, the present invention provides compounds of formula (III), compound of formula (IV), compound of formula (V), compound of formula (VI), or stereoisomers or pharmaceutically acceptable salts or solvates thereof:

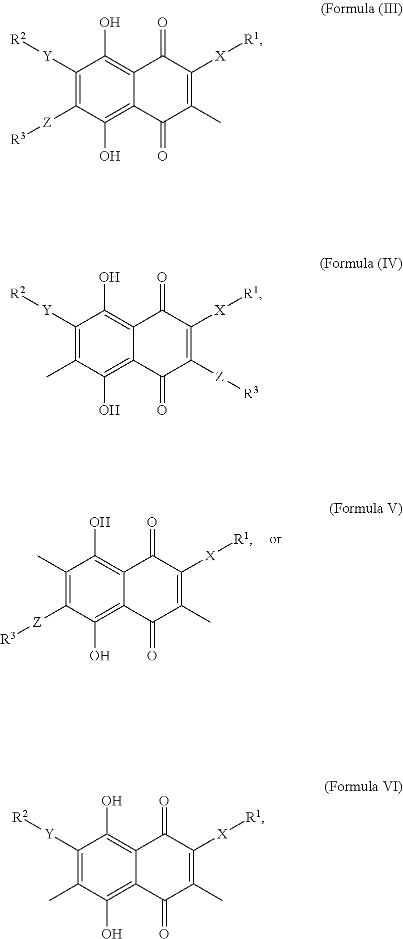

wherein
X, Y and Z independently represent hydrogen, —C(R⁴)—, —O—, —S—, —CH₂O—, CH₂S—, —(CH₂)₂O—, —NR⁵—, —NR⁵CH₂—, —CH₂NR⁵—, —NR⁵CO—, —CONR⁵—, —N═N—, —NH—CO—NH—, —NH—CS—NH—, —CO—O—, CO—O—CH₂—, —SO₂NH—, —NH—SO₂—, —CR⁴═CR⁴—, —C≡C—, —O—CH₂—CO—, —OCH₂CH₂O—, —CH(OH)—, or —NO₂ bridging groups;
R⁴ independently represent hydrogen, halogen, C₁₋₆ alkyl, C₁₋₆ alkenyl, C₁₋₆ alkoxy, C₁₋₆ haloalkyl, haloC₁₋₆ alkoxy, —COOH, —CONH₂, —COC₁₋₆ alkyl, O—C₁₋₆ alkyl, NH₂, NH—C₁₋₆ alkyl, or —SC₁₋₆ alkyl groups; and
R¹, R², R³, and R⁵ independently represent hydrogen, halogen, aryl, C₃₋₈ cycloalkyl, monocyclic or bicyclic heterocyclyl, monocyclic or bicyclic heteroaryl, wherein the aryl, heteroaryl or heterocyclyl groups may be optionally substituted by one or more R⁴ groups.

In an embodiment, the disclosure provides for a pharmaceutical composition comprising at least one compound of formula (II), (III), (IV), (V), or (VI) or a pharmaceutically acceptable salt or solvate thereof. In an embodiment, the pharmaceutical compound is for use in treatment of a proliferative disease, such as a cancer, for example, a breast cancer. A further embodiment may provide a method of treating breast cancer comprising administering to a subject a compound according to any one of the preceding paragraphs. The breast cancer may be an ER-positive breast cancer and/or a HER2-positive breast cancer. The subject may express a mutant ER-α protein and/or a HER2Δ16 and/or presence of truncated HER2 (p95HER2). An embodiment may provide use of a compound as in the paragraphs above for treating breast cancer. In some embodiments the breast cancer is an ER-positive breast cancer or HER2 positive breast cancer. In some embodiments said subject expresses a mutant ER-α protein and/or a HER2Δ16 and/or presence of truncated HER2 (p95HER2). In some embodiments a compound as presented above is used in the preparation of a medicament for treatment of breast cancer.

The pharmaceutical compositions of the present disclosure can be in any form known to those of skill in the art. For instance, in some embodiments the pharmaceutical compositions are in a form of a product for oral delivery, said product form being selected from a group consisting of a concentrate, dried powder, liquid, capsule, pellet, and pill. In other embodiments, the pharmaceutical compositions of the disclosure are in the form of a product for parenteral administration including intravenous, intradermal, intramuscular, and subcutaneous administration. The pharmaceutical compositions disclosed herein may also further comprise carriers, binders, diluents, and excipients.

Also, in other aspects, the present disclosure relates to a tyrosine kinase inhibitor composition comprising one or more compounds selected from the group consisting of a compound of Formula (I), (II), (III), (IV), (V), and (VI) and its derivatives, and pharmaceutically acceptable salts and solvates thereof. In an embodiment, said compound has a purity of ≥75%, ≥80%, ≥85%, ≥90%, ≥95%, ≥96%, ≥97%, or ≥98%, and ≥99%. In an embodiment, a pharmaceutical composition is provided comprising the claimed tyrosine kinase inhibitor composition, either alone or in combination with at least one additional therapeutic agent, with a pharmaceutically acceptable carrier; and uses of the claimed tyrosine kinase inhibitor compositions, either alone or in combination with at least one additional therapeutic agent, in the treatment of proliferative diseases including breast cancer at any stage of the disease diagnosis. The combination with an additional therapeutic agent may take the form of combining the claimed tyrosine kinase inhibitor compounds with any known therapeutic agent.

The methods for treating a clinical indication by the tyrosine kinase inhibitor compounds disclosed herein, may be effectuated by administering a therapeutically effective amount of the tyrosine kinase inhibitor compounds to a patient in need thereof, this therapeutically effective amount may comprise administration of the prodrug to the patient at 1 mg/kg/day, 2 mg/kg/day, 3 mg/kg/day, 4 mg/kg/day, 5 mg/kg/day, 10 mg/kg/day and 20 mg/kg/day. Alternatively, amounts ranging from about 0.001 mg/kg/day to about 0.01 mg/kg/day, or about 0.01 mg/kg/day to about 0.1 mg/kg/day, or about 0.1 mg/kg/day to about 1 mg/kg/day, or about 1 mg/kg/day to 10 mg/kg/day, or about 10 mg/kg/day to about 100 mg/kg/day are also contemplated.

A further object of the disclosure is a kit, comprising a composition containing at least one tyrosine kinase inhibitor compounds disclosed herein for treatment and prevention of cancer and cancer related morbidities. The composition of the kit may comprise at least one carrier, at least one binder, at least one diluent, at least one excipient, at least one other therapeutic agent, or mixtures thereof.

One aspect of the present disclosure is the compounds disclosed herein as well as the intermediates as used for their synthesis.

While certain features of this invention shown and described below are pointed out in the annexed claims, the invention is not intended to be limited to the details specified, since a person of ordinary skill in the relevant art will understand that various omissions, modifications, substitutions, and changes in the forms and details of the invention illustrated and in its operation may be made without departing in any way from the spirit of the present invention. No feature of the invention is critical or essential unless it is expressly stated as being “critical” or “essential.”

These and other features, aspects, and advantages of embodiments of the present disclosure will become better understood with regard to the following descriptions, claims, and accompanying drawings explained below.

### BRIEF DESCRIPTION OF THE DRAWINGS

For a further understanding of the nature, objects, and advantages of the present disclosure, reference should be had to the following detailed description, read in conjunction with the following drawings, wherein like reference numerals denote like elements.

FIG. 1 shows a scheme for synthesis of 5,8-dihydroxynapthalene-1,4-dione derivatives using Friedel Crafts acylation.

FIG. 2 shows another scheme for synthesis of 5,8-dihydroxynaphthalene-1,4-dione derivatives and 5-methylnaphthalene-1,4-dione.

FIG. 3 shows another scheme for synthesis of 5-hydroxy-8-methylnaphthalene-1,4-dione and 5-methylnaphthalene-1,4-dione.

FIG. 4 shows another scheme for synthesis of 5-hydroxy-7-methylnaphthalene-1,4-dione, 5-methoxy-methylnaphthalene-1,4-dione, and 5-hydroxy-7-bromomethylnaphthalene-1,4-dione.

FIG. 5 shows charts of cell viability after 48 hr treatment of each compound at a concentration of 10 μM.

FIG. 6 shows dose response curves for inhibition of MCF-7/pcDNA, MCF-7/HER2 and MCF-7/HER2Δ16 cells by compounds 7 (A), 9 (B), and (8a+8b) (C).

FIG. 7 shows western blot analysis of autophosphorylation at the HER2 residue Y1248. Compounds 6, 7, (8a+8b) and 9 inhibit the phosphorylation at HER2-Y1248 in MCF7/pcDNA, MCF/HER2 and MCF7/HER216. Compounds 15, 25, 26 and 27, did not show notable inhibition of phosphorylation at HER2-Y1248 even though they showed notable inhibition in the high-throughput assay. Compound 28 (2-methyl-4,6-dinitro-1H-indene-1,3(2H)-dione), which did not show notable inhibition in the high-throughput assay shows significant phosphorylation at HER2-Y1248 in all of the three cell lines.

FIG. 8 shows Western blot analysis of autophosphorylation at the EGFR residue Y1068. Compounds 6, 7, (8a+8b) and 9 inhibit the phosphorylation at EGFR-Y1068 in MCF7/pcDNA, MCF/HER2 and MCF7/HER216. Compound 26 did not show notable inhibition of phosphorylation at HER2-Y1248 even though it showed notable inhibition in the high-throughput assay. Compound 28 (2-methyl-4,6-dinitro-1H-indene-1,3(2H)-dione), which did not show notable inhibition in the high-through put assay shows significant phosphorylation at HER2-Y1248 and EGFR-Y1068 in all of the three cell lines.

FIG. 9 shows inhibition of various breast cancer cell lines by compound 7.

FIG. 10 shows high throughput assay of Compound 7 against a panel of 100 disease relevant kinases at a concentration of 10 μM performed by Thermo Fisher Scientific's SelectScreen™ Profiling Service.

FIG. 11(A) shows ATP-binding pocket of HER2 kinase domain created using X-ray structures. The molecular surface depiction of the binding pocket is based on lipophilic and lipophilic characteristics. The dimensions of the two linked cavities in the ATP-binding pocket is shown. FIG. 11(B) shows the structure of planar compound 7 and its dimensions. FIG. 11(C) shows binding mode of compound 7 in the inner second cavity of the ATP-binding pocket of HER2 kinase domain. FIG. 11(D) shows the hydrogen bonding interaction of compound 7 with the ATP-binding pocket residues Thr862, Leu796 and Leu852.

### DETAILED DESCRIPTION

Before the subject disclosure is further described, it is to be understood that the disclosure is not limited to the particular embodiments of the disclosure described below, as variations of the particular embodiments may be made and still fall within the scope of the appended claims. It is also to be understood that the terminology employed is for the purpose of describing particular embodiments, and is not intended to be limiting. Instead, the scope of the present disclosure will be established by the appended claims.

In this specification and the appended claims, the singular forms “a,” “an,” and “the” include plural reference unless the context clearly dictates otherwise. Unless defined otherwise, all technical and scientific terms used herein have the same meaning as commonly understood to one of ordinary skill in the art to which this disclosure belongs.

As used herein, the term “minimize” or “reduce”, or derivatives thereof, include a complete or partial inhibition of a specified biological effect (which is apparent from the context in which the terms “minimize” or “reduce” are used).

The compounds according to the disclosure are isolated and purified in a manner known per se, e.g. by distilling off the solvent in vacuo and recrystallizing the residue obtained from a suitable solvent or subjecting it to one of the customary purification methods, such as chromatography on a suitable support material. Furthermore, reverse phase preparative HPLC of compounds of the present disclosure which possess a sufficiently basic or acidic functionality, may result in the formation of a salt, such as, in the case of a compound of the present disclosure which is sufficiently basic, a trifluoroacetate or formate salt for example, or, in the case of a compound of the present disclosure which is sufficiently acidic, an ammonium salt for example. Salts of this type can either be transformed into its free base or free acid form, respectively, by various methods known to the person skilled in the art, or be used as salts in subsequent biological assays. Additionally, the drying process during the isolation of compounds of the present disclosure may not fully remove traces of cosolvents, especially such as formic acid or trifluoroacetic acid, to give solvates or inclusion complexes. The person skilled in the art will recognize which solvates or inclusion complexes are acceptable to be used in subsequent biological assays. It is to be understood that the specific form (e.g., salt, free base, solvate, inclusion complex) of a compound of the present disclosure as isolated as described herein is not necessarily the only form in which said compound can be applied to a biological assay in order to quantify the specific biological activity.

One aspect of the disclosure is salts of the compounds according to the disclosure including all inorganic and organic salts, especially all pharmaceutically acceptable inorganic and organic salts, particularly all pharmaceutically acceptable inorganic and organic salts customarily used in pharmacy.

Examples of salts include, but are not limited to, lithium, sodium, potassium, calcium, aluminum, magnesium, titanium, meglumine, ammonium, salts optionally derived from NH₃ or organic amines having from 1 to 16 C-atoms such as, e.g., ethylamine, diethylamine, triethylamine, ethyldiisopropylamine, monoethanolamine, diethanolamine, triethanolamine, dicyclohexylamine, dimethylaminoethanol, procaine, dibenzylamine, N-methylmorpholine, arginine, lysine, ethylenediamine, N-methylpiperindine and guanidinium salts.

The salts include water-insoluble and, particularly, water-soluble salts.

As used herein, “pharmaceutically acceptable salts” refer to derivatives of the compounds disclosed herein wherein the parent compound is modified by making acid or base salts thereof. Examples of pharmaceutically acceptable salts include, but are not limited to, mineral or organic acid salts of basic residues such as amines, alkali or organic salts of acidic residues such as carboxylic acids, and the like. The pharmaceutically acceptable salts include the conventional non-toxic salts or the quaternary ammonium salts of the parent compound formed, for example, from non-toxic inorganic or organic acids. For example, such conventional non-toxic salts include, but are not limited to, those derived from inorganic and organic acids selected from 2-acetoxybenzoic, 2-hydroxyethane sulfonic, acetic, ascorbic, benzene sulfonic, benzoic, bicarbonic, carbonic, citric, edetic, ethane disulfonic, 1,2-ethane sulfonic, fumaric, glucoheptonic, gluconic, glutamic, glycolic, glycollyarsanilic, hexylresorcinic, hydrabamic, hydrobromic, hydrochloric, hydroiodic, hydroxymaleic, hydroxynaphthoic, isethionic, lactic, lactobionic, lauryl sulfonic, maleic, malic, mandelic, methane sulfonic, napsylic, nitric, oxalic, pamoic, pantothenic, phenylacetic, phosphoric, polygalacturonic, propionic, salicyclic, stearic, subacetic, succinic, sulfamic, sulfanilic, sulfuric, tannic, tartaric, toluene sulfonic, and the commonly occurring amine acids, e.g., glycine, alanine, phenylalanine, arginine, etc.

Other examples of pharmaceutically acceptable salts include hexanoic acid, cyclopentane propionic acid, pyruvic acid, malonic acid, 3-(4-hydroxybenzoyl)benzoic acid, cinnamic acid, 4-chlorobenzenesulfonic acid, 2-naphthalenesulfonic acid, 4-toluenesulfonic acid, camphorsulfonic acid, 4-methylbicyclo-[2.2.2]-oct-2-ene-1-carboxylic acid, 3-phenylpropionic acid, trimethylacetic acid, tertiary butylacetic acid, muconic acid, and the like. The present disclosure also encompasses salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like. In the salt form, it is understood that the ratio of the compound to the cation or anion of the salt may be 1:1, or any ratio other than 1:1, e.g., 3:1, 2:1, 1:2, or 1:3.

It should be understood that all references to pharmaceutically acceptable salts include solvent addition forms (solvates) or crystal forms (polymorphs) as defined herein, of the same salt.

Salts of the compounds of formulas (I), (II), (III), (IV), (V), or (VI) according to the disclosure can be obtained by dissolving the free compound in a suitable solvent (for example a ketone such as acetone, methylethylketone or methylisobutylketone, an ether such as diethyl ether, tetrahydrofuran or dioxane, a chlorinated hydrocarbon such as methylene chloride or chloroform, or a low molecular weight aliphatic alcohol such as methanol, ethanol or isopropanol) which contains the desired acid or base, or to which the desired acid or base is then added. The acid or base can be employed in salt preparation, depending on whether a mono- or polybasic acid or base is concerned and depending on which salt is desired, in an equimolar quantitative ratio or one differing therefrom. The salts are obtained by filtering, reprecipitating, precipitating with a non-solvent for the salt or by evaporating the solvent. Salts obtained can be converted into the free compounds which, in turn, can be converted into salts. In this manner, pharmaceutically unacceptable salts, which can be obtained, for example, as process products in the manufacturing on an industrial scale, can be converted into pharmaceutically acceptable salts by processes known to the person skilled in the art.

According to the person skilled in the art the compounds of formulas (I) through (VI) according to this disclosure as well as their salts may contain, e.g., when isolated in crystalline form, varying amounts of solvents. Included within the scope of the disclosure are therefore all solvates and in particular all hydrates of the compounds of formulas (I) through (VI) according to this disclosure as well as all solvates and in particular all hydrates of the salts of the compounds of formulas (I) through (VI) according to this disclosure.

“Solvate” means solvent addition forms that contain either stoichiometric or non stoichiometric amounts of solvent. Some compounds have a tendency to trap a fixed molar ratio of solvent molecules in the crystalline solid state, thus forming a solvate. If the solvent is water the solvate formed is a hydrate; and if the solvent is alcohol, the solvate formed is an alcoholate. Hydrates are formed by the combination of one or more molecules of water with one molecule of the substance in which the water retains its molecular state as H₂O.

The compounds according to the disclosure and their salts can exist in the form of tautomers which are included in the embodiments of the disclosure.

“Tautomer” is one of two or more structural isomers that exist in equilibrium and is readily converted from one isomeric form to another. This conversion results in the formal migration of a hydrogen atom accompanied by a switch of adjacent conjugated double bonds. Tautomers exist as a mixture of a tautomeric set in solution. In solutions where tautomerization is possible, a chemical equilibrium of the tautomers will be reached. The exact ratio of the tautomers depends on several factors, including temperature, solvent and pH. The concept of tautomers that are interconvertible by tautomerizations is called tautomerism.

Where the present specification depicts a compound prone to tautomerization, but only depicts one of the tautomers, it is understood that all tautomers are included as part of the meaning of the chemical depicted. It is to be understood that the compounds disclosed herein may be depicted as different tautomers. It should also be understood that when compounds have tautomeric forms, all tautomeric forms are intended to be included, and the naming of the compounds does not exclude any tautomer form.

Of the various types of tautomerism that are possible, two are commonly observed. In keto-enol tautomerism a simultaneous shift of electrons and a hydrogen atom occurs. Ring-chain tautomerism arises as a result of the aldehyde group (—CHO) in a sugar chain molecule reacting with one of the hydroxy groups (—OH) in the same molecule to give it a cyclic (ring-shaped) form as exhibited by glucose.

Common tautomeric pairs are: ketone-enol, amide-nitrile, lactam-lactim, amide-imidic acid tautomerism in heterocyclic rings (e.g., in nucleobases such as guanine, thymine and cytosine), imine-enamine and enamine-enamine.

The compounds of the disclosure may, depending on their structure, exist in different stereoisomeric forms. These forms include configurational isomers or optically conformational isomers (enantiomers and/or diastereoisomers including those of atropisomers). The present disclosure therefore includes enantiomers, diastereoisomers as well as mixtures thereof. From those mixtures of enantiomers and/or diastereoisomers pure stereoisomeric forms can be isolated with methods known in the art, preferably methods of chromatography, especially high performance liquid chromatography (HPLC) using achiral or chiral phase. The disclosure further includes all mixtures of the stereoisomers mentioned above independent of the ratio, including the racemates.

The compounds of the disclosure may, depending on their structure, exist in various stable isotopic forms. These forms include those in which one or more hydrogen atoms have been replaced with deuterium atoms, those in which one or more nitrogen atoms have been replaced with ¹⁵N atoms, or those in which one or more atoms of carbon, fluorine, chlorine, bromine, sulfur, or oxygen have been replaced by the stable isotope of the respective, original atoms.

Some of the compounds and salts according to the disclosure may exist in different crystalline forms (polymorphs) which are within the scope of the disclosure.

It is a further object of the disclosure to provide tyrosine kinase inhibitor compounds disclosed herein, methods of synthesizing the tyrosine kinase inhibitor compounds, methods of manufacturing the tyrosine kinase inhibitor compounds, and methods of using the tyrosine kinase inhibitor compounds. The compounds can also be made by synthetic schemes well established in the art.

Another object of the disclosure is to provide a composition, for example a pharmaceutical composition, comprising at least one tyrosine kinase inhibitor compound disclosed herein in an amount effective for the indication of proliferative diseases such as cancer, including but not limited to endocrine related cancer. In an embodiment, the cancer is an ER-positive tumor and/or a HER2-positive tumor, such as a tumor of the breast, endometrium, uterus, or ovary. In an embodiment, the tumor is an ER-positive and/or HER2-positive tumor of the breast. In an embodiment, the breast tumor is determined to be ER-positive by an immunohistochemical method described by Hammond et al.

In an embodiment, the object of such treatment is to inhibit tyrosine kinases.

As used herein, “treating” means administering to a subject a pharmaceutical composition to ameliorate, reduce or lessen the symptoms of a disease. As used herein, “treating” or “treat” describes the management and care of a subject for the purpose of combating a disease, condition, or disorder and includes the administration of a compound disclosed herein, or a pharmaceutically acceptable salt, polymorph or solvate thereof, to alleviate the symptoms or complications of a disease, condition or disorder, or to eliminate the disease, condition or disorder. The term “treat” may also include treatment of a cell in vitro or an animal model. As used herein, “subject” or “subjects” refers to any animal, such as mammals including rodents (e.g., mice or rats), dogs, primates, lemurs or humans.

Treating cancer may result in a reduction in size of a tumor. A reduction in size of a tumor may also be referred to as “tumor regression.” Preferably, after treatment, tumor size is reduced by 5% or greater relative to its size prior to treatment; more preferably, tumor size is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75% or greater. Size of a tumor may be measured by any reproducible means of measurement. The size of a tumor may be measured as a diameter of the tumor.

Treating cancer may result in a reduction in tumor volume. Preferably, after treatment, tumor volume is reduced by 5% or greater relative to its size prior to treatment; more preferably, tumor volume is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75% or greater. Tumor volume may be measured by any reproducible means of measurement.

Treating cancer may result in a decrease in number of tumors. Preferably, after treatment, tumor number is reduced by 5% or greater relative to number prior to treatment; more preferably, tumor number is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75%. Number of tumors may be measured by any reproducible means of measurement. The number of tumors may be measured by counting tumors visible to the naked eye or at a specified magnification. Preferably, the specified magnification is 2×, 3×, 4×, 5×, 10×, or 50×.

Treating cancer may result in a decrease in number of metastatic lesions in other tissues or organs distant from the primary tumor site. Preferably, after treatment, the number of metastatic lesions is reduced by 5% or greater relative to number prior to treatment; more preferably, the number of metastatic lesions is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75%. The number of metastatic lesions may be measured by any reproducible means of measurement. The number of metastatic lesions may be measured by counting metastatic lesions visible to the naked eye or at a specified magnification. Preferably, the specified magnification is 2×, 3×, 4×, 5×, 10×, or 50×.

Treating cancer may result in an increase in average survival time of a population of treated subjects in comparison to a population receiving carrier alone. Preferably, the average survival time is increased by more than 30 days; more preferably, by more than 60 days; more preferably, by more than 90 days; and most preferably, by more than 120 days. An increase in average survival time of a population may be measured by any reproducible means. An increase in average survival time of a population may be measured, for example, by calculating for a population the average length of survival following initiation of treatment with an active compound. An increase in average survival time of a population may also be measured, for example, by calculating for a population the average length of survival following completion of a first round of treatment with an active compound.

Treating cancer may result in an increase in average survival time of a population of treated subjects in comparison to a population of untreated subjects. Preferably, the average survival time is increased by more than 30 days; more preferably, by more than 60 days; more preferably, by more than 90 days; and most preferably, by more than 120 days. An increase in average survival time of a population may be measured by any reproducible means. An increase in average survival time of a population may be measured, for example, by calculating for a population the average length of survival following initiation of treatment with an active compound. An increase in average survival time of a population may also be measured, for example, by calculating for a population the average length of survival following completion of a first round of treatment with an active compound.

Treating cancer may result in increase in average survival time of a population of treated subjects in comparison to a population receiving monotherapy with a drug that is not a compound disclosed herein, or a pharmaceutically acceptable salt thereof. Preferably, the average survival time is increased by more than 30 days; more preferably, by more than 60 days; more preferably, by more than 90 days; and most preferably, by more than 120 days. An increase in average survival time of a population may be measured by any reproducible means. An increase in average survival time of a population may be measured, for example, by calculating for a population the average length of survival following initiation of treatment with an active compound. An increase in average survival time of a population may also be measured, for example, by calculating for a population the average length of survival following completion of a first round of treatment with an active compound.

Treating cancer may result in a decrease in the mortality rate of a population of treated subjects in comparison to a population receiving carrier alone. Treating cancer may result in a decrease in the mortality rate of a population of treated subjects in comparison to an untreated population. Treating cancer may result in a decrease in the mortality rate of a population of treated subjects in comparison to a population receiving monotherapy with a drug that is not a compound disclosed herein, or a pharmaceutically acceptable salt, prodrug, metabolite, analog or derivative thereof. Preferably, the mortality rate is decreased by more than 2%; more preferably, by more than 5%; more preferably, by more than 10%; and most preferably, by more than 25%. A decrease in the mortality rate of a population of treated subjects may be measured by any reproducible means. A decrease in the mortality rate of a population may be measured, for example, by calculating for a population the average number of disease-related deaths per unit time following initiation of treatment with an active compound. A decrease in the mortality rate of a population may also be measured, for example, by calculating for a population the average number of disease-related deaths per unit time following completion of a first round of treatment with an active compound.

Treating cancer may result in a decrease in tumor growth rate. Preferably, after treatment, tumor growth rate is reduced by at least 5% relative to number prior to treatment; more preferably, tumor growth rate is reduced by at least 10%; more preferably, reduced by at least 20%; more preferably, reduced by at least 30%; more preferably, reduced by at least 40%; more preferably, reduced by at least 50%; even more preferably, reduced by at least 50%; and most preferably, reduced by at least 75%. Tumor growth rate may be measured by any reproducible means of measurement. Tumor growth rate may be measured according to a change in tumor diameter per unit time.

Treating cancer may result in a decrease in tumor regrowth, for example, following attempts to remove it surgically. Preferably, after treatment, tumor regrowth is less than 5%; more preferably, tumor regrowth is less than 10%; more preferably, less than 20%; more preferably, less than 30%; more preferably, less than 40%; more preferably, less than 50%; even more preferably, less than 50%; and most preferably, less than 75%. Tumor regrowth may be measured by any reproducible means of measurement. Tumor regrowth is measured, for example, by measuring an increase in the diameter of a tumor after a prior tumor shrinkage that followed treatment. A decrease in tumor regrowth is indicated by failure of tumors to reoccur after treatment has stopped.

Treating or preventing a cell proliferative disorder may result in a reduction in the rate of cellular proliferation. Preferably, after treatment, the rate of cellular proliferation is reduced by at least 5%; more preferably, by at least 10%; more preferably, by at least 20%; more preferably, by at least 30%; more preferably, by at least 40%; more preferably, by at least 50%; even more preferably, by at least 50%; and most preferably, by at least 75%. The rate of cellular proliferation may be measured by any reproducible means of measurement. The rate of cellular proliferation is measured, for example, by measuring the number of dividing cells in a tissue sample per unit time.

Treating or preventing a cell proliferative disorder may result in a reduction in the proportion of proliferating cells. Preferably, after treatment, the proportion of proliferating cells is reduced by at least 5%; more preferably, by at least 10%; more preferably, by at least 20%; more preferably, by at least 30%; more preferably, by at least 40%; more preferably, by at least 50%; even more preferably, by at least 50%; and most preferably, by at least 75%. The proportion of proliferating cells may be measured by any reproducible means of measurement. Preferably, the proportion of proliferating cells is measured, for example, by quantifying the number of dividing cells relative to the number of nondividing cells in a tissue sample. The proportion of proliferating cells may be equivalent to the mitotic index.

Treating or preventing a cell proliferative disorder may result in a decrease in size of an area or zone of cellular proliferation. Preferably, after treatment, size of an area or zone of cellular proliferation is reduced by at least 5% relative to its size prior to treatment; more preferably, reduced by at least 10%; more preferably, reduced by at least 20%; more preferably, reduced by at least 30%; more preferably, reduced by at least 40%; more preferably, reduced by at least 50%; even more preferably, reduced by at least 50%; and most preferably, reduced by at least 75%. Size of an area or zone of cellular proliferation may be measured by any reproducible means of measurement. The size of an area or zone of cellular proliferation may be measured as a diameter or width of an area or zone of cellular proliferation.

Treating or preventing a cell proliferative disorder may result in a decrease in the number or proportion of cells having an abnormal appearance or morphology. Preferably, after treatment, the number of cells having an abnormal morphology is reduced by at least 5% relative to its size prior to treatment; more preferably, reduced by at least 10%; more preferably, reduced by at least 20%; more preferably, reduced by at least 30%; more preferably, reduced by at least 40%; more preferably, reduced by at least 50%; even more preferably, reduced by at least 50%; and most preferably, reduced by at least 75%. An abnormal cellular appearance or morphology may be measured by any reproducible means of measurement. An abnormal cellular morphology may be measured by microscopy, e.g., using an inverted tissue culture microscope. An abnormal cellular morphology may take the form of nuclear pleiomorphism.

### EXAMPLES

Hereby are provided non-limiting examples of embodiments of compounds disclosed herein.

### Example

5,8-dihydroxynaphthoquinone derivatives substituted at the 2, 3, 6 and 7 positions were synthesized. Four different schemes were employed for this purpose.

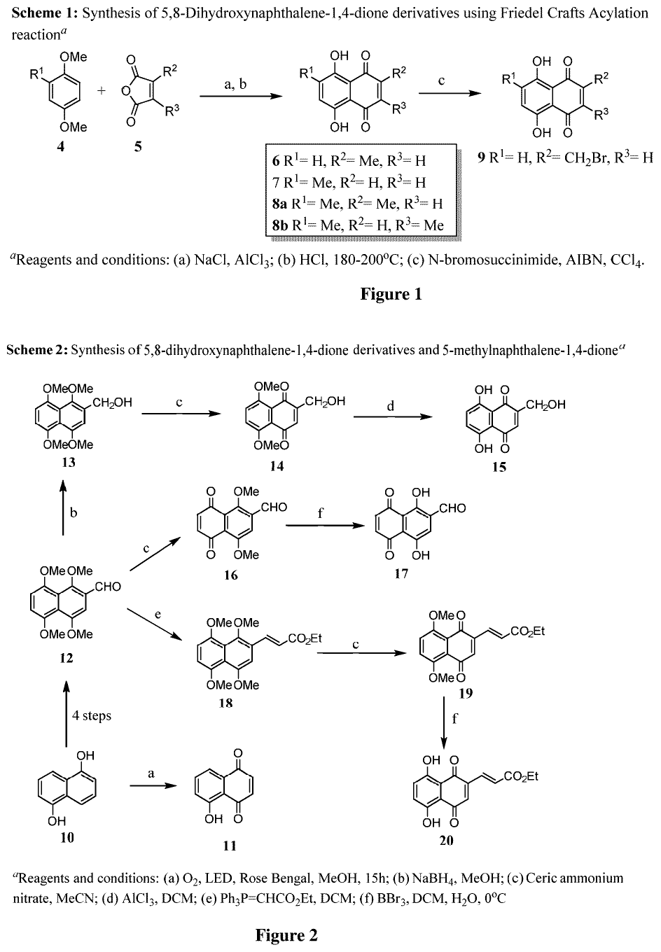

Scheme 1 (FIG. 1) involved the double Friedel-Crafts acylation reaction using substituted maleic anhydrides and/or substituted 1,4-dimethoxybenzene in the presence of aluminum chloride and sodium chloride at high temperatures followed by demethylation with HCl at 0° C. (25-55% yield). With a methyl substitution on both of the reactants 8a and 8b isomeric mixture of products were obtained as inseparable mixture. With the use of visible light initiation (120 W flood lamps), free radical halogenation of compound 6 using N-bromosuccinimide gave the brominated side chain compound 9 in 32% yield.

The second synthetic route (FIG. 2) made use of the established reaction sequence for generating 1,4,5,8-tetramethoxy-2-naphthaldehyde from 1,5-dihydroxynaphthalene 12. Compounds 14, 15, 16 and 17 were prepared using reported methods. The preparation of ethyl (E)-3-(5,8-dihydroxy-1,4-dioxo-1,4-dihydronaphthalen-2-yl)acrylate 20 was achieved through a three step process involving the Wittig reaction of 2-formyl-1,4,5,8-tetramethoxynaphthalene 12 and ethyl (triphenylphosphoranylidene)acetate in DCM at room temperature. The phosphonium ylide was prepared through nucleophilic substitution of ethyl 2-bromoacetate with triphenylphosphine under refluxing conditions in toluene. The precipitated phophonium bromide salt was filtered, then deprotonated with sodium hydroxide to afford ethyl (triphenylphosphoranylidene)acetate. The obtained Wittig product ethyl (E)-3-(1,4,5,8-tetramethoxynaphthalen-2-yl)acrylate 18 was then oxidized using aqueous ammonium cerium (IV) nitrate at room temperature which afforded a mixture of isomers that were easily separated through silica gel chromatography and showed distinct chemical shifts in proton NMR. Demethylation of ethyl (E)-3-(5,8-dimethoxy-1,4-dioxo-1,4-dihydronaphthalen-2-yl)acrylate was achieved through a boron tribromide adduct which was hydrolyzed by addition of water to afford the product 20 (41% yield from 12). The photooxidation method was used to prepare 5-hydroxynaphthalene-1,4-dione (juglone) 11 from 1,5-dihydroxynaphthalene by bubbling molecular oxygen in methanol in the presence rose-bengal catalyst and green LED.

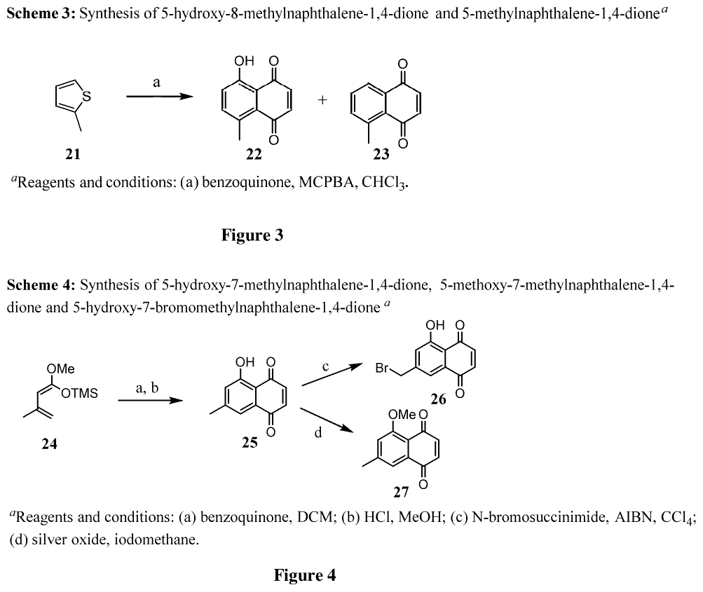

Compounds 5-hydroxy-8-methylnaphthalene-1,4-dione 22 and 5-methylnaphthalene-1,4-dione 23 were prepared through a Diels-Alder [4+2] cycloaddition of 2-methylthiophene and 1,4-benzoquinone, with m-chloroperoxybenzoic acid in chloroform for 48 h followed by purification using silica gel chromatography in FIG. 3.

Compound 5-hydroxy-7-methylnaphthalene-1,4-dione 25 was prepared through a Diels-Alder [4+2] cycloaddition using 3-methyl-1-methoxy-1-trimethylsiloxy-1,4-diene and 1,4-benzoquinone as a dienophile at 0° C. for 20 h in FIG. 4. The resulting 7-methyl 5-(trimethylsiloxy)naphthalene-1,4-dione adduct was hydrolyzed using 1N HCl in methanol to afforded the product 25. Compound 5-methoxy-7-methylnaphthalene-1,4-dione 27 was obtained by refluxing 25 in silver (II) oxide with iodomethane followed by filtration over celite and flash chromatography. Side chain bromination of compound 25 to form compound 26 was accomplished using N-bromosuccinimide in the presence of the radical initiator AIBN in carbon tetrachloride under refluxing conditions.

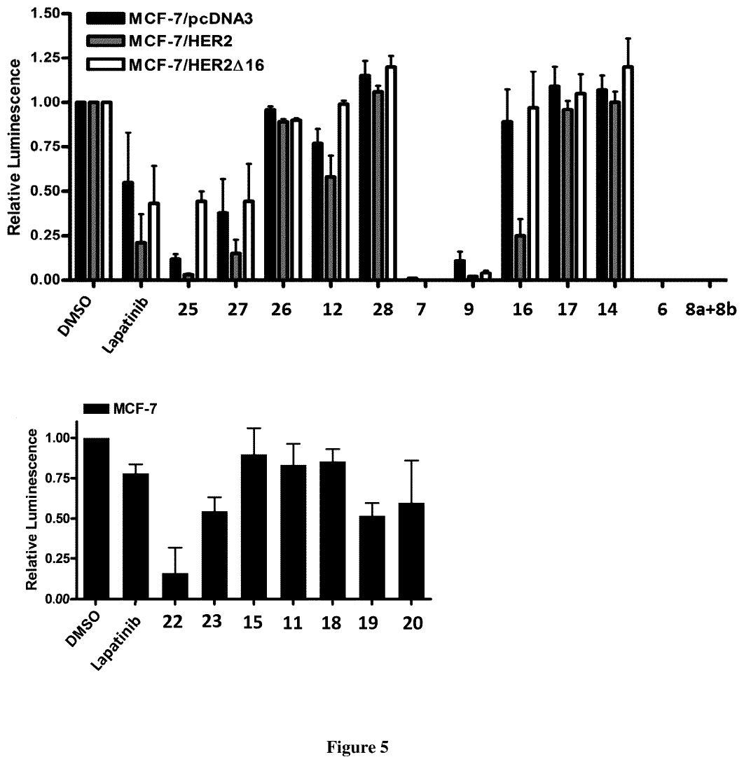

The antagonistic activities of the compounds were tested in an estrogen receptor positive (ER+) breast cancer cell line, MCF7 breast cancer cell lines expressing HER2, HER2Δ16 and empty vector. Lapatinib was used as the positive control. The cells were treated with the compounds at 10 μM concentration for 48 h. After treatment, cell viability was tested using the CellTiter-Glo Assay (Promega). Out of a total of 24 compounds mentioned above tested, 5 compounds suppressed cell viability potently in all three cell lines. After 48 h, these 5 compounds decreased cell viability by >90% when compared to lapatinib with a cell viability of <70% at the same concentration. 8 compounds showed moderate suppression of cell viability with many of them showing a greater inhibition of the MCF7-HER2 cell line than the MCF7-HER2Δ16 cell line in a pattern that was similar to that lapatinib (FIG. 5). The six best compounds 7, 9, the mixture of 8a+8b, 25 and 27 were then taken up for further studies.

The IC₅₀ values, defined as the concentration of the compound required to inhibit cell proliferation by 50%, of the six best compounds from the high-throughput screening were measured by treating each of the cell lines with different drug concentrations for 48 h followed by the CellTiter-Glo Assay to detect cell viability (Table 1).

TABLE 1
Inhibition of MCF-7/pcDNA, MCF-7/HER2 and MCF-7/HER2Δ16
breast cancer cell lines by the six compounds identified
from the high-throughput assay.

| | IC₅₀ in μM | | |
|---|---|---|---|
| Compound | MCF-7/pcDNA | MCF-7/HER2 | MCF-7/HER2D16 |
| 7 | 0.32 | 0.29 | 0.51 |
| 8a + 8b | 1.28 | 1.30 | 0.51 |
| 9 | 1.66 | 1.78 | 3.61 |
| 22 | 2.93 | 4.61 | 9.39 |
| 25 | 30.88 | ND | ND |
| 27 | 4.94 | 3.18 | 7.30 |

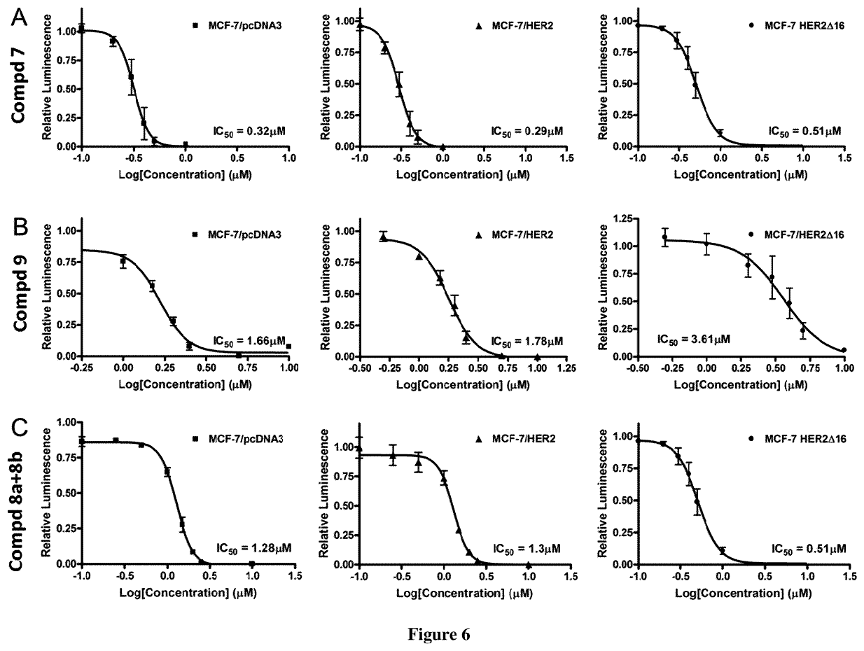

Lapatinib, a known HER2/EGFR inhibitor in clinical use was taken as the positive standard. Lapatinib inhibited the three cell lines MCF-7/pcDNA, MCF-7/HER2 and MCF-7/HER2Δ16 with an IC₅₀ value of 15.71 μM, 15.79 μM and 19.22 μM. Other than compound 25, most of our compounds (7, 9, the mixture of 8a+8b, and 27) showed higher potency of inhibition of the three breast cancer cell lines than lapatinib. The dose-response curves for the three compounds 7, the mixture of 8a+8b and 9 with the lowest IC₅₀ values for MCF-7/pcDNA (0.32 M, 1.28 μM and 1.66 μM), MCF-7/HER2 (0.29 μM, 1.30 μM and 1.78 μM) and MCF-7/HER2D16 (0.51 μM, 0.51 μM and 3.61 μM) are represented in FIG. 6. Compound 7 showed the lowest inhibition potency for all three cell lines.

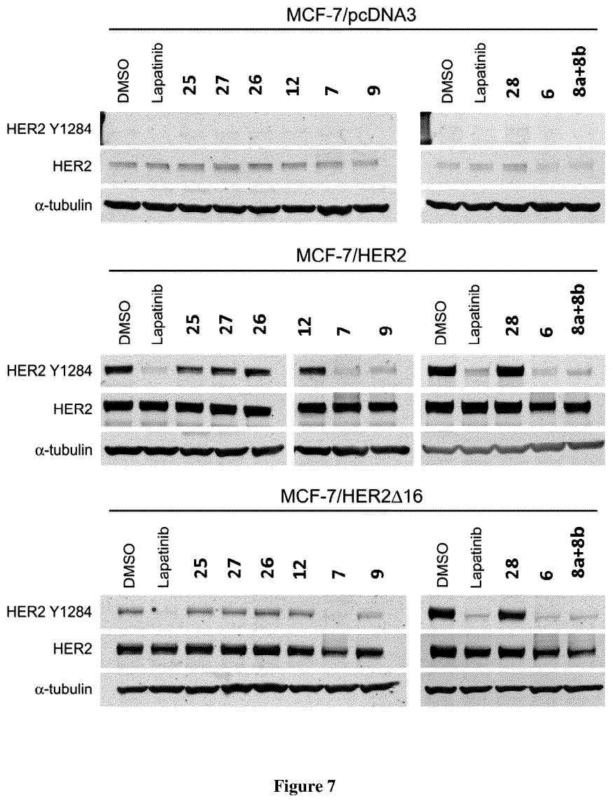

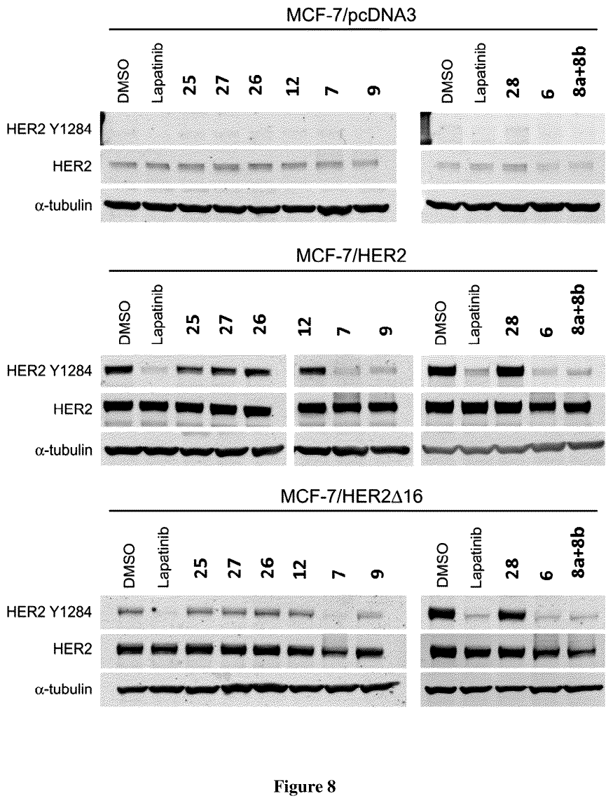

HER2 is a member of the erbB family of tyrosine kinases, which is comprised of four partial homologous transmembrane receptors: EGFR/HER1 (erbB1), HER2 (erbB2/neu), HER3 (erbB3), and HER4 (erbB4). With the exception of HER2, these other receptors exhibit ligand specificity. Ligand binding induces homo- or hetero-dimerization through disulfide bond linkage, and leads to receptor activation and tyrosine auto-phosphorylation. HER2 is a preferred dimerization partner due to its high catalytic activity and forms potent heterodimers with EGFR and HER3. HER2, with its intrinsic kinase activity can transphosphorylate other members of the erbB family. HER2 and HERΔ16 are overexpressed in the breast cancer cell lines that have been used in this study, leading to the kinases being constitutively active. Upon dimerization, the constitutively active HER2 and HER2Δ16 receptors can transphosphorylate coexpressed EGFR. The levels of phosphorylated HER2 in the parental MCF-7 cell line were less, resulting in its lack of dimerization with EGFR and its transphosphorylation. Western blots were performed to determine the ability of the compounds to inhibit the activation of the HER2 receptor (FIG. 7). The total and phosphorylated protein were detected upon treatment of each of the cells lines with the compounds 6, 7, 8a+8b, 9, 15, 25, 26, 27 and 28 (2-methyl-4,6-dinitro-1H-indene-1,3(2H)-dione) along with the positive control lapatinib at 10 μM concentration for 2 h. Compound 28 (2-methyl-4,6-dinitro-1H-indene-1,3(2H)-dione) did not show notable growth inhibition in the high-throughput assay and was used as a negative control. Compounds 6, 7, 8a+8b, and 9 significantly decreased HER2 activating phosphorylation to the same extent as lapatinib at the auto-phosphorylation site Y1284. Compounds 15, 25, 26, and 27 did not show such an effect on auto-phosphorylation. The ability of these compounds to inhibit the breast cancer cell lines and not the HER2 auto-phosphorylation at Y1284 indicates that the mechanism of growth inhibition in cancer cells might involve some other alternate pathway. The compounds 6, 7, 8a+8b, and 9 that showed decreased autophosphorylation in HER2 were then tested for the ability to decrease transphosphorylation of the EGFR receptor at the residue Y1068 (FIG. 8). Compounds 26 and 28 from FIG. 5 were used as negative controls. The transphosphorylation at Y1068 of EGFR receptor was decreased by the compounds 6, 7, 8a+8b, and 9 in levels comparable to that of lapatinib.

### Compound 7 Example

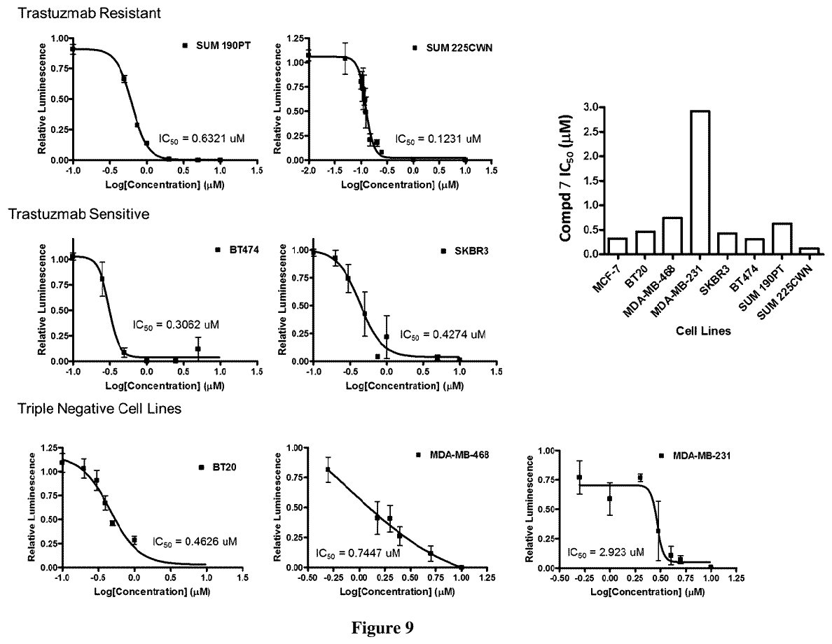

Compound 7 was taken up for further analysis of its ability to inhibit the growth of a panel of breast cancer cell lines of varied types. The panel included the triple negative cell lines BT20, MDA-MB-468 and MDA-MB-231, the trastuzumab sensitive cell lines SKBR3 and BT474, and the trastuzumab resistant cell lines SUM 190PT and SUM 225CWN (FIG. 9). Compound 7 inhibited the growth of these breast cancer cell lines with IC₅₀ values ranging from 0.1231 μM to 2.923 μM. The potency of inhibition was comparable for most of the cell lines that were tested except for MDA-MB-231 where a 3-4 fold decrease in potency was evidenced.

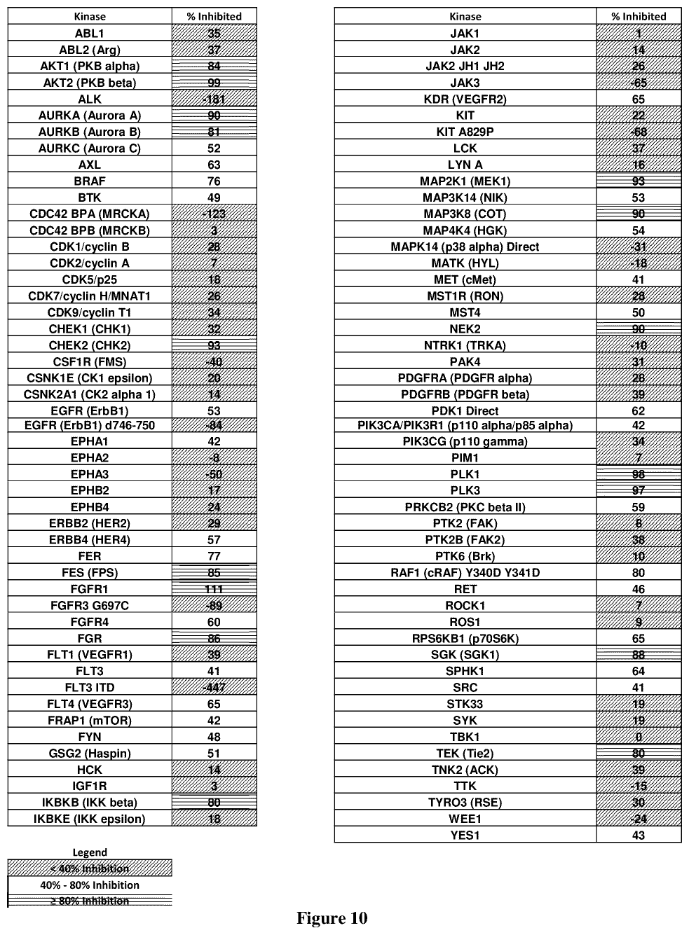

Compound 7 was then subjected to a cross kinase panel high-throughput assay at 10 μM concentration, at Life Technologies, to determine the selectivity of kinase inhibition (FIG. 10). A panel of 120 disease relevant kinases (available with Life Technologies) were studied. 11 kinases were inhibited at 80% or more-protein kinase B 1/2 (also known as AKT1/2), Aurora kinases A/B (AurK A/B), checkpoint kinase 2 (CHEK2), feline sarcoma kinase (FES), fibroblast growth factor 1 (FGFR1), I kappa B kinase B/E (IKK B/E), mitogen activated protein kinase kinase 1 (MAP2K1), mitogen activated protein kinase kinase kinase 8 (MAP3K8), Never in Mitosis (NIMA) related kinase 2 (NEK2), polo-like kinase 1/3 (PLK1/3), serum/glucocorticoid regulated kinase 1 (SGK1) and TEK tyrosine kinase. All these kinases except for CHEK2 are reported to have tumorigenic roles in breast cancer [13-20].

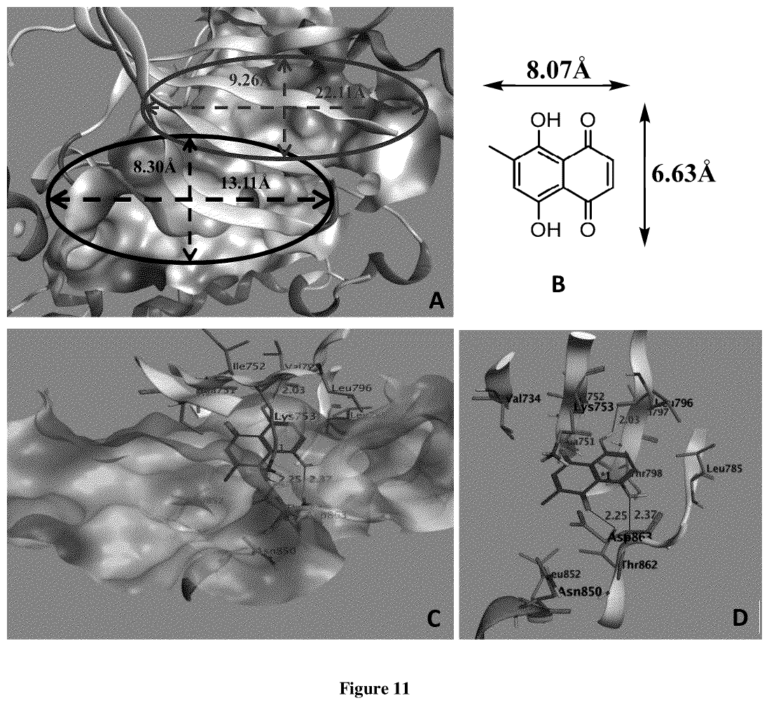

Docking studies were performed on the active compounds with the HER2 X-ray crystal structure using the Molecular Operating Environment (MOE) software's docking module. The binding pocket of the HER2 protein is L-shaped with two large cavities that are connected to each other (FIG. 11(A)). The cavity where the base of the ATP molecule binds to the hinge region has a width of ˜13.11 Å and height of 8.30 Å. The second cavity behind the first cavity has the invariant Lys753 and Asp853 residues outlining it with a width of 22.13 Å and height of 9.20 Å. Compound 7 is a planar molecule with the dimensions 8.07 Å X 6.63 Å (FIG. 11(B)). Docking studies of this compound revealed that it preferred to reside in the second cavity where it made more hydrogen bonds with the sidechain hydroxyl group of Thr862, backbone —NH of Asp863 and the backbone carbonyl of Leu796 (FIG. 11(C) and FIG. 11(D)). The sidechain methyl group of compound 7 depicted hydrophobic interactions with the sidechain of Leu852 and the sidechain methyl group of Thr862. On the other hand, compound 3 with the structure

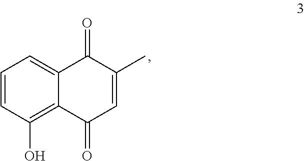

with one less phenolic group on the other hand preferred to reside in the first cavity. The additional hydrogen bonds made by the compound 7 might have contributed to its greater efficacy in growth inhibition of the breast cancer cell lines MCF-7/HER2 and MCF-7/HER2Δ16.

With several of the highly selective kinase inhibitors in clinical settings as breast cancer therapeutics leading to the development of resistance within a year, the thought arises whether inhibition of more than one key tumorigenic kinase would be the path to take for achieving success in the fight against aggressive and refractory breast cancer. Such an approach will also require better success at targeted delivery of these agents to minimize any side effects due to multiple kinase targeting. The compounds of the present invention may solve these needs.

All references cited in this specification are herein incorporated by reference as though each reference was specifically and individually indicated to be incorporated by reference. The citation of any reference is for its disclosure prior to the filing date and should not be construed as an admission that the present disclosure is not entitled to antedate such reference by virtue of prior invention.

It will be understood that each of the elements described above, or two or more together may also find a useful application in other types of methods differing from the type described above. Without further analysis, the foregoing will so fully reveal the gist of the present disclosure that others can, by applying current knowledge, readily adapt it for various applications without omitting features that, from the standpoint of prior art, fairly constitute essential characteristics of the generic or specific aspects of this disclosure set forth in the appended claims. The foregoing embodiments are presented by way of example only; the scope of the present disclosure is to be limited only by the following claims.

### REFERENCES CITED

1. https://www.cancer.net/cancer-types/breast-cancer/statistics
2. https://www.cancer.gov/news-events/cancer-currents-blog/2015/breast-forecast
3. https://www.bcrf.org/breast-cancer-statistics
4. http://chemoth.com/types/kinaseinhibitors
5. https://www.webmd.com/breast-cancer/guide/breast-cancer-types-er-positive-her2-positive#1
6. https://www.ncbi.nlm.nih.gov/pmc/articles/PMC5267957/
7. https://www.healthline.com/health/breast-cancer/her2-positive-survival-rates-statistics#survival-rates
8. https://www.webmd.com/breast-cancer/guide/breast-cancer-types-er-positive-her2-positive#2
9. https://www.ncbi.nlm.nih.gov/pmc/articles/PMC5004067/
10. https://www.ncbi.nlm.nih.gov/pmc/articles/PMC5004067/
11. Hudis, C. A., Trastuzumab—mechanism of action and use in clinical practice. N Engl J Med 2007, 357 (1), 39-51.
12. Gatzemeier, U.; Groth, G.; Butts, C.; Van Zandwijk, N.; Shepherd, F.; Ardizzoni, A.; Barton, C.; Ghahramani, P.; Hirsh, V., Randomized phase II trial of gemcitabine-cisplatin with or without trastuzumab in HER2-positive non-small-cell lung cancer. Ann Oncol 2004, 15 (1), 19-27.
13. Riggio, M.; Perrone, M. C.; Polo, M. L.; Rodriguez, M. J.; May, M.; Abba, M.; Lanari, C.; Novaro, V., AKT1 and AKT2 isoforms play distinct roles during breast cancer progression through the regulation of specific downstream proteins. Sci Rep 2017, 7, 44244.
14. Tang, A.; Gao, K.; Chu, L.; Zhang, R.; Yang, J.; Zheng, J., Aurora kinases: novel therapy targets in cancers. Oncotarget 2017, 8 (14), 23937-23954.
15. Siveen, K. S.; Prabhu, K. S.; Achkar, I. W.; Kuttikrishnan, S.; Shyam, S.; Khan, A. Q.; Merhi, M.; Dermime, S.; Uddin, S., Role of Non Receptor Tyrosine Kinases in Hematological Malignances and its Targeting by Natural Products. Mol Cancer 2018, 17 (1), 31.
16. Perez-Garcia, J.; Munoz-Couselo, E.; Soberino, J.; Racca, F.; Cortes, J., Targeting FGFR pathway in breast cancer. Breast 2018, 37, 126-133.
17. Boehm, J. S.; Zhao, J. J.; Yao, J.; Kim, S. Y.; Firestein, R.; Dunn, I. F.; Sjostrom, S. K.; Garraway, L. A.; Weremowicz, S.; Richardson, A. L.; Greulich, H.; Stewart, C. J.; Mulvey, L. A.; Shen, R. R.; Ambrogio, L.; Hirozane-Kishikawa, T.; Hill, D. E.; Vidal, M.; Meyerson, M.; Grenier, J. K.; Hinkle, G.; Root, D. E.; Roberts, T. M.; Lander, E. S.; Polyak, K.; Hahn, W. C., Integrative genomic approaches identify IKBKE as a breast cancer oncogene. Cell 2007, 129 (6), 1065-79.
18. Tang, X.; Jin, L.; Cao, P.; Cao, K.; Huang, C.; Luo, Y.; Ma, J.; Shen, S.; Tan, M.; Li, X.; Zhou, M., MicroRNA-16 sensitizes breast cancer cells to paclitaxel through suppression of IKBKB expression. Oncotarget 2016, 7 (17), 23668-83.
19. O'Shea, J.; Cremona, M.; Morgan, C.; Milewska, M.; Holmes, F.; Espina, V.; Liotta, L.; O'Shaughnessy, J.; Toomey, S.; Madden, S. F.; Carr, A.; Elster, N.; Hennessy, B. T.; Eustace, A. J., A preclinical evaluation of the MEK inhibitor refametinib in HER2-positive breast cancer cell lines including those with acquired resistance to trastuzumab or lapatinib. Oncotarget 2017, 8 (49), 85120-85135.
20. Fang, Y.; Zhang, X., Targeting NEK2 as a promising therapeutic approach for cancer treatment. Cell Cycle 2016, 15 (7), 895-907.

What is claimed is:

1. A compound according to Formula (II), and/or a stereoisomer and/or pharmaceutically acceptable salt and/or solvate thereof: 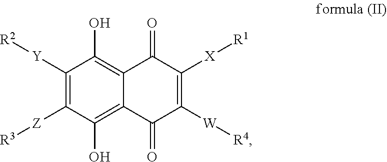
wherein:
W, X, Y and Z independently represent hydrogen, —C(R⁵)—, —O—, —S—, —CH₂O—, CH₂S—, —(CH₂)₂O—, —NR⁶—, —NR⁶CH₂—, —CH₂NR⁶—, —NR⁶CO—, —CONR⁶—, —N═N—, —NH—CO—NH—, —NH—CS—NH—, —CO—O—, CO—O—CH₂—, —SO₂NH—, —NH—SO₂—, —CR⁴═CR⁴—, —C≡C—, —O—CH₂—CO—, —OCH₂CH₂O—, —CH(OH)—, or —NO₂ bridging groups;
R⁵ independently represent hydrogen, halogen, C₁₋₆ alkyl, C₁₋₆ alkenyl, C₁₋₆ alkoxy, C₁₋₆haloalkyl, haloC₁₋₆alkoxy, —COOH, —CONH₂, —COC₁₋₆ alkyl, O—C₁₋₆ alkyl, NH₂, NH—C₁₋₆ alkyl, or —SC₁₋₆ alkyl groups; and
R¹, R², R³, R⁴ and R⁶ independently represent hydrogen, halogen, aryl, C₃₋₈ cycloalkyl, monocyclic or bicyclic heterocyclyl, monocyclic or bicyclic heteroaryl, wherein the aryl, heteroaryl or heterocyclyl groups may be optionally substituted by one or more R⁴ groups.

2. The compound according to claim 1,
wherein
W represents —C(R⁵)—,
R⁵ represents hydrogen, and
R⁴ represents hydrogen.

3. The compound according to claim 1,
wherein
Z represents —C(R⁵)—,
R⁵ represents hydrogen, and
R³ represents hydrogen.

4. The compound according to claim 1,
wherein
W and Y represent —C(R⁵)—,
R⁵ represents hydrogen, and
R² and R⁴ represent hydrogen.

5. The compound according to claim 1,
wherein
W and Z represent —C(R⁵)—,
R⁵ represents hydrogen, and
R³ and R⁴ represent hydrogen.

6. The compound according to claim 1, wherein at least one of W, X, Y, or Z is methyl, and R¹, R², R³, and R⁴ are hydrogen.

7. The compound according to claim 1, wherein
Y, X, and W are independently selected from H or —C(R⁵)—;
Z is hydrogen;
R¹, R², R³, and R⁴ are independently selected from hydrogen or halogen, and
R⁵ is hydrogen.

8. The compound according to claim 1, wherein
Y X, and W are independently selected from hydrogen or methyl;
Z is hydrogen; and
R¹, R², R³, and R⁴ are independently selected from hydrogen or halogen.

9. The compound according to claim 1, wherein said compound is 5,8-dihydroxy-2-methylnaphthalene-1,4-dione.

10. The compound according to claim 1, wherein said compound is 5, 8-dihydroxy-6-methylnaphthalene-1,4-dione.

11. The compound according to claim 1, wherein said compound is 5,8-dihydroxy-2,7-dimethylnaphthalene-1,4-dione.

12. The compound according to claim 1, wherein said compound is 5,8-dihydroxy-2,6-dimethylnaphthalene-1,4-dione.

13. The compound according to claim 1, wherein said compound is 2-(bromomethyl)-5,8-dihydroxynaphthalene-1,4-dione.

14. The compound of claim 1 for use in the treatment of a proliferative disease in a mammal in need thereof.

15. The compound of claim 1 for use in the treatment of a cancer in a mammal in need thereof.

16. The compound of claim 1 for use in inhibiting a tyrosine kinase to treat a tyrosine kinase-dependent disease in a mammal in need thereof.

17. A composition comprising the compound of claim 1 for use as a medicament.

18. A pharmaceutical composition comprising a compound, pharmaceutically acceptable salt, solvate, or composition of claim 1 and a pharmaceutically acceptable carrier.

19. The pharmaceutical composition of claim 18, suitable for enteral administration.

20. The pharmaceutical composition of claim 18, wherein said pharmaceutical composition is suitable for oral administration.

21. The pharmaceutical composition of claim 18, suitable for parenteral administration.

22. A method of treating a tyrosine kinase dependent disease comprising administering to a subject a compound, pharmaceutically acceptable salt, or solvate of claim 1 or a pharmaceutical composition thereof.

23. A method of treating breast cancer comprising administering to a subject in need of such treatment a compound, pharmaceutically acceptable salt, or solvate of claim 1 or a pharmaceutical composition thereof.

24. The method of claim 23, wherein said breast cancer is an ER-positive breast cancer.

25. The method of claim 23, where said subject expresses a mutant ER-α protein.

26. The method of claim 23, wherein said breast cancer is an HER2-positive breast cancer.

27. The method of claim 23, wherein said subject expresses a HER2Δ16.

28. The method of claim 23, wherein said subject expresses a truncated HER2 (p95HER2).
